(19) United States
(12) Patent Application Publication
(10) Pub. No.: US 2026/0263671 A1
(43) Pub. Date: Sep. 10, 2026

(54) SYSTEM FEATURING OPTICAL AND ELECTRICAL SENSORS FOR CHARACTERIZING EFFLUENT FROM A PERITONEAL DIALYSIS PATIENT

(71) Applicants: VANTIVE US HEALTHCARE LLC, Deerfield, IL (US); VANTIVE HEALTH GMBH, Glattpark (Opfikon) (CH)

(72) Inventors: James Patrick McCanna, Pleasanton, CA (US); Erik Edwin Tang, San Diego, CA (US); Marshal Singh Dhillon, San Diego, CA (US); Matthew John Banet, San Diego, CA (US); Justin Grant Buckingham, Oceanside, CA (US); Shawn Collin Oppegard, Fox River Grove, IL (US); Mattias Holmer, Lund (SE)

(21) Appl. No.: 19/166,391

(22) PCT Filed: Mar. 27, 2024
(86) PCT No.: PCT/EP2024/058184

§ 371 Date: Sep. 17, 2025

Related U.S. Application Data
(63) Continuation of application No. 18/194,286, filed on Mar. 31, 2023.

(51) Int. Cl. A61M 1/28

(52) U.S. Cl. CPC A61M 1/28; A61M 2205/3306; A61M 2205/3331

(57) ABSTRACT

The invention provides a system for characterizing an effluent sample from a patient undergoing peritoneal dialysis. The system features a container for enclosing the effluent sample, and optical system, an electrical system, and a processor. The optical system features a light source and a photodetector, the light source emitting a beam of radiation that passes through the container and irradiates the effluent sample, and the photodetector detecting the radiation after it irradiates the effluent sample to generate an optical signal. The electrical system typically features a first pair of electrodes and a second pair electrodes, both attached directly to the container and arranged to measure a capacitance of the effluent sample to generate a capacitance signal. The processor operates an algorithm that collectively processes the optical and capacitance signals to characterize the effluent sample.

### TECHNICAL FIELD

The present disclosure relates to systems for monitoring patients, specifically those suffering from end stage renal disease (herein “ESRD”) and undergoing peritoneal dialysis (herein “PD”), in both hospital and home environments.

### BACKGROUND

PD is a type of dialysis that uses the peritoneum in a patient's abdomen as the membrane through which fluid and dissolved substances are exchanged with the blood. Typically PD is used to remove excess fluid, correct electrolyte problems, and remove toxins from patients suffering from ESRD. PD is typically less efficient at removing wastes from the body than hemodialysis (herein “HD”). However, PD typically has better outcomes than HD during the first few of years of deployment. Compared to HD. PD allows greater patient mobility, produces fewer swings in symptoms due to its continuous nature, and is better at removing phosphate compounds. However PD also typically removes large amounts of albumin, which in turn requires constant monitoring of the patient's nutritional status. Costs associated with PD are generally lower than those of HD in most parts of the world; this is most apparent in developed economies. PD's other benefits include greater flexibility and better tolerability for patients with significant heart disease.

PD may either occur at regular intervals throughout the day, known as continuous ambulatory peritoneal dialysis (herein “CAPD”), or at night with the assistance of a machine, known as automated peritoneal dialysis machine (herein “APD”) or ‘cycler’. The solution is typically made of sodium chloride, hydrogen carbonate, and an osmotic agent such as glucose.

During PD, a dialysate solution (also denoted dialysate or dialysis fluid herein) is introduced through a permanent catheter that is deployed in the patient's lower abdomen during a surgical procedure. The catheter is inserted with one end in the abdomen and the other protruding from the skin. Not surprisingly, the presence of the catheter presents a risk of peritonitis because it can introduce bacteria to the abdomen. Typically 2-3 liters of dialysate solution is introduced into the abdomen at the start of PD therapy. The volume, referred to as a ‘fill volume’, can be as much as 3 liters, and medication can also be added to the solution immediately before infusion. The volume remains in the abdomen and waste products diffuse across the peritoneum from the underlying blood vessels. After a variable period of time, the “dwell time” (usually 2-6 hours depending on the treatment), the fluid is removed—the removed fluid is called the ‘effluent’. Removal of the effluent can occur automatically while the patient is sleeping (e.g., during APD), or during the day by keeping 2 liters of fluid in the abdomen at all times, exchanging the fluids 4-6 times per day (CAPD). APD cycles between 3-10 dwells per night, while CAPD involves 4 dwells per day of 2-3 liters per dwell, with each remaining in the abdomen for 4-8 hours.

PD effluent can be monitored to determine if the patient is suffering from early onset of peritonitis, or some other deleterious condition that may, for example, impact the color of the effluent. For example, a pink-tinged effluent suggests bleeding inside the abdomen or menstruation, while the presence of a brown or yellow tint in the effluent may indicate feces, which in turn suggests a perforated bowel. A cloudy effluent typically suggests infection. Often the test for determining this condition is holding a waste bag that contains the effluent up to a normally readable document (e.g., a magazine or newspaper), and determining if the text can be read. A document that cannot be clearly read indicates a cloudy effluent, which in turn may indicate the presence of leukocytes (e.g., white blood cells) or other biological matter and thus peritonitis. As such, the evaluation of PD effluent is a manual process that is difficult to repeat with any meaningful accuracy. The subjectivity of the process, requiring visual evaluation by a particularly individual, further affects the overall accuracy of this process.

Some automated systems have been developed for evaluating PD effluent. These include, for example, optical systems based on optical absorption, scattering, or fluorescence to characterize PD effluent; others describe measurements of the effluent's chemical properties, such as pH. In still other examples, systems in the prior art measure physiological properties from the patient (e.g., blood pressure, glucose) to estimate their condition, e.g., the presence of peritonitis. Issued patents in this area include, for example: U.S. Pat. Nos. 11,013,843; 10,983,124; 10,925,549; 10,758,659; 10,744,253; 10,537,673; 10,155,081; 10,010,289; 9,518,914; 9,215,985; 9,125,989; 8,945,936; and 8,801,652.

In view of the foregoing, it would be beneficial to improve upon conventional approaches for monitoring PD effluent from ESRD patients, with the goal of determining conditions such as peritonitis as early as possible.

### SUMMARY

The technical solution described herein provides an automated, quantitative measurement system for detecting leukocytes and other biological matter in PD effluent. In this way, the technical solution enables detection of infections and other conditions in the patient's abdomen—e.g., peritonitis, bleeding, presence of feces—in its early stage, thus allowing clinicians to intervene and provide appropriate measures (e.g., prescription of antibiotics). Ultimately, this means the system may help identify such infections and allow them to be addressed and possibly ameliorated, thus allowing the patient to continue PD therapy and avoid HD therapy. This can be advantageous, as HD is typically relatively costly, uncomfortable, and inconvenient for the patient. The system that performs these measurements—referred to herein as a PD effluent analysis system (herein “PDEAS”)—can feature optical, impedance, and other sensors that measure PD effluent either directly in a PD machine, or alternatively in tubing that drains PD effluent from the patient (into, e.g., a toilet). The PDEAS is a compact, low-cost system that requires little or no interaction from the user. After measuring time-dependent waveforms with its various sensors, the PDEAS analyzes these data and transmits information directly to the APD (e.g., for dynamic control by the APD); additionally or alternatively, the PDEAS can transmit information to the cloud, either directly (e.g., through an internal cellular modem or Wi-Fi chipset) or through a user's device (e.g., mobile phone, tablet computer). There, algorithms can analyze the information, facilitate early detection of peritonitis and other health conditions, and pave the way for early clinical actions before these conditions worsen.

Given the above, in one aspect, the present disclosure relates to a system for characterizing an effluent sample from a patient undergoing PD. The system typically features a container for enclosing the effluent sample, and optical system, an electrical system, and a processor. The optical system features a light source and a photodetector, wherein the light source emits a beam of radiation that passes through the container and irradiates the effluent sample, and the photodetector detects the radiation (e.g., scattered radiation) after it irradiates the effluent sample to generate an optical signal. The electrical system features at least one pair of electrodes, which is attached to the container and arranged to measure an electrical signal (e.g., capacitance) of the effluent sample to generate a measured electrical response (e.g., capacitance signal). For example, the processor operates an algorithm that collectively processes the optical and electrical signals to characterize the effluent sample. It should be appreciated, however, that other electrical signals (besides capacitance) are likewise contemplated herein, as disclosed in greater detail below. The at least one pair of electrodes may be directly or indirectly attached to the container.

In embodiments, the container is a sample cell that includes at least two surfaces. Each of these surfaces of the sample cell can include an optically transparent material, such as a material consisting of glass, plastic, ceramic, diamond-based material, or derivatives thereof. Typically the electrode is a thin film, and ideally a thin film that is both optically transparent and electrically conductive, that is deposited on at least one of the two surfaces. In an embodiment, for example, the thin film is composed primarily of gold. In other embodiment, the thin film includes In₂O₅Sn or derivatives thereof (e.g., indium tin oxide, herein “ITO”). Alternatively the thin film is composed of a thin metal that has reasonable transparency to optical wavelengths in the infrared, visible, and ultraviolet spectral ranges.

In embodiments, both the first and second surfaces of the sample cell include optically transparent electrodes. Here, the light source is configured to emit a beam of radiation that passes through a first optically transparent electrode and into the effluent sample, and the photodetector is configured to receive the radiation after it irradiates the effluent sample and then passes through a second optically transparent electrode. An electrical system that integrates with such a system can feature a capacitor that includes two capacitor electrodes, and the first optically transparent electrode is a first capacitor electrode, and the second optically transparent electrode is a second capacitor electrode.

In related embodiments, the electrical system comprises a first pair of electrodes and a second pair of electrodes, where both the first and second pair of electrodes comprise an optically transparent opening (e.g., a cut-out region in an otherwise continuous, conductive region), as opposed to being fully optically transparent. Here, the light source is configured to emit a beam of radiation that passes through a first optically transparent opening in a first electrode and into the effluent sample, and the photodetector is configured to receive the radiation after it irradiates the effluent sample and then passes through a second optically transparent opening in a second electrode.

In related embodiments, the optical system is further configured to measure an optical absorption of the effluent sample, e.g., a multi-frequency absorption spectrum. Here, the light source is configured to emit a beam of radiation that passes into the effluent sample, the photodetector is configured to receive the radiation after it irradiates the effluent sample, and the processor is configured to analyze the radiation after it irradiates the effluent sample and determine the amount of radiation absorbed by the effluent sample. In this and other embodiments, the photodetector, for example, can be a standard photodiode, CCD camera, a photodiode coupled to a computer-controlled optical filter, or several closely packed photodiodes with different optical filters.

In other related embodiments, the optical system is further configured to measure an optical scattering caused by the effluent sample. Here, the light source is configured to emit a beam of radiation that passes into the effluent sample, the photodetector is configured to receive the radiation after it irradiates the effluent sample, and the processor is configured to analyze the radiation after it irradiates the effluent sample and determine the amount of optical scattering caused by the effluent sample.

In still other embodiments, the electrical system is further configured to measure one or more electrical parameters, such as capacitance, resistance, conductivity, complex impedance, impedance, reactance, inductance, permittivity, magnetism, and other related properties of the effluent sample. To make such a measurement, for example, the first electrode, taken alone or combined with another electrode such as the second electrode, is further configured to both induce electrical current into the effluent sample either directly via direct current or by creating an electrical field in the effluent sample via alternating current, and then sense the electrical property or a parameter related thereto of the effluent sample. Here, for example, the electrical system may induce different currents into the sample, each characterized by a different frequency (typically ranging between 5-1000 kHz). Measuring signals resulting from these induced currents results in a spectrum of electrical properties (e.g., an impedance or capacitance spectrum) that, in turn, can be analyzed to determine compounds within the effluent sample.

In related embodiments, the electrical system further includes both third and fourth electrodes. Here, for example, the system includes a first set (pair) of electrodes featuring two electrodes that are configured to induce electrical current into the effluent sample, and a second set (pair) of electrodes, separate from the first set of electrodes, that are configured to sense the electrical property of the effluent sample.

In other embodiments, the processor used in the system is further configured to operate an algorithm that collectively processes the optical signal and the electrical signal to determine an amount of a compound (e.g., leukocytes, blood cells, proteins, such as fibrine, fat particles, triglycerides, chylomicrons, micelles, biological materials, and derivatives thereof) in the effluent sample. The processor can be included directly on the system and within the PD machine. Alternatively, the processor may be included in the cloud, or in a device connected to the PD machine, such as a computer, tablet computer, or mobile device.

Most typically the system described herein is integrated directly with an electromechanical cycler used for PD. For example, it can be incorporated directly within the electromechanical PD cycler, e.g., it can be integrated with (or actually be a part of) a portion of a tube within the electromechanical cycler (e.g., an internal tubing component). Alternatively, the system is connected to an external tube, external to the cycler (or is actually part of the second tube) that connects to the patient and is normally used to drain effluent fluid from the patient's peritoneal cavity.

In another aspect, the present disclosure relates to a system similar to that above, in which the electrical system features first and second pair of electrodes, with both electrode pairs attached to the container and arranged to measure an electrical property other than capacitance (e.g., resistance, conductivity, complex impedance, impedance, reactance, inductance, permittivity, magnetism, and related properties) of the effluent sample. Here, the processor operates an algorithm that collectively processes the optical and electrical signals to characterize the effluent sample.

In an aspect, the system's electrodes are optically transparent. In another aspect, a first pair of electrodes induce an electrical current into the effluent sample, and a second pair of electrodes measure an electrical signal of the effluent sample that depends on the electrical current that is induced into the sample; in a different aspect, a single pair of electrodes is configured to both (1) induce an electrical current into the effluent sample and (2) measure an electrical signal of the effluent sample that depends on the electrical current that is induced into the sample. And in yet another aspect, the system can be used to characterize any liquid sample, and not just PD effluent.

The proposed technique features several advantages. Most importantly, combining multiple optical and additional electrical measurement techniques into a single measurement system may improve the sensitivity of the system, thereby allowing it, for example, to detect small amounts of leukocytes in PD effluent. In theory this means that peritonitis in a PD patient can be detected at a very early stage, thereby allowing a clinician to intervene with therapeutic measures (e.g., administration of antibiotics) to stave off the infection. Ultimately, this could allow an ESRD patient to stay with PD, which as described above has several advantages compared to HD. Additionally, by incorporating multiple measurement techniques into a single system, the proposed technique involves sensing different physiological components of the PD effluent, thus reducing the probably of false readings—e.g., ‘false negatives’ and ‘false positives’—that can negatively impact clinical intervention. More specifically, optical measurements, and particularly optical absorption spectroscopy, are sensitive to the specific molecular structure of the particulate matter. Such measurements can be complimented by electrical techniques, such as multi-frequency measurement of parameters including impedance, reactance, resistance, and capacitance, which are very sensitive markers of the presence of particulate matter in a PD effluent. Additionally, such measurements can be complemented by optical measurements, and particularly optical absorption spectroscopy, which are sensitive to the specific molecular structure of the particulate matter. Taken in combination, these techniques provide an effective way to evaluate PD effluent while avoiding false negatives and false positives that may otherwise compromise this measurement.

In light of the disclosure here, and without limiting the scope in any way, a first aspect of the present disclosure is a system for characterizing an effluent sample from a patient undergoing peritoneal dialysis (PD). The system includes a container, an optical system, an electrical system, and a processor. The container encloses the effluent sample. The optical system includes a light source and a photodetector, the light source configured to emit a beam of radiation that passes through the container and irradiates the effluent sample, and the photodetector configured to detect the radiation after it irradiates the effluent sample to generate an optical signal. The electrical system includes at least one pair of electrodes. The at least one pair of electrodes is attached to the container and configured to measure an electrical property of the effluent sample to generate a property signal. The processor operates an algorithm configured to collectively process the optical signal and the property signal to characterize the effluent sample.

In the following, embodiments of the first aspect are presented. Each such embodiment may be combined with any other embodiment listed herein unless specified otherwise.

In one embodiment, the container is a sample cell comprising at least two surfaces.

In one embodiment, each of the at least two surfaces of the sample cell comprises an optically transparent material.

In one embodiment, the optically transparent material is selected from a group consisting of glass, plastic, ceramic, diamond-based material, or derivatives thereof.

In one embodiment, the at least one pair of electrodes is a thin film deposited on at least one of the two surfaces.

In one embodiment, the thin film is a material that is both optically transparent and electrically conductive.

In one embodiment, the thin film is composed primarily of one of gold. In₂O₅Sn. or derivatives thereof.

In one embodiment, the at least one pair of electrodes comprises a first pair of electrodes and a second pair of electrodes.

In one embodiment, a first surface of the container comprises the first pair of electrodes, which is a first optically transparent electrode pair. The second surface of the container comprises the second pair of electrodes, which is a second optically transparent electrode pair.

In one embodiment, the light source is configured to emit the beam of radiation that passes through the first optically transparent electrode pair and into the effluent sample. The photodetector is configured to receive the radiation after it irradiates the effluent sample and then passes through the second optically transparent electrode pair.

In one embodiment, the electrical system comprises a capacitor comprising two capacitor electrodes. The first optically transparent electrode pair is a first capacitor electrode pair, and the second optically transparent electrode pair is a second capacitor electrode pair.

In one embodiment, both the first pair of electrodes and the second pair of electrodes comprise an optically transparent opening.

In one embodiment, the light source is configured to emit the beam of radiation that passes through a first optically transparent opening in the first pair of electrodes and into the effluent sample. The photodetector is configured to receive the radiation after it irradiates the effluent sample and then passes through a second optically transparent opening in the second pair of electrodes.

In one embodiment, the optical system is further configured to measure an optical absorption of the effluent sample.

In one embodiment, the light source is configured to emit the beam of radiation that passes into the effluent sample, the photodetector is configured to receive the radiation after it irradiates the effluent sample, and the processor is configured to analyze the radiation after it irradiates the effluent sample and determine the amount of radiation absorbed by the effluent sample.

In one embodiment, the optical system is further configured to measure an optical scattering caused by the effluent sample.

In one embodiment, the light source is configured to emit the beam of radiation that passes into the effluent sample, the photodetector is configured to receive the radiation after it irradiates the effluent sample, and the processor is configured to analyze the radiation after it irradiates the effluent sample and determine the amount of optical scattering caused by the effluent sample.

In one embodiment, the electrical property of the effluent sample is selected from the group consisting of resistance, conductivity, complex impedance, impedance, reactance, inductance, permittivity, and magnetism.

In one embodiment, the at least one pair of electrodes comprises a pair of electrodes that is configured to both induce electrical current into the effluent sample and sense the electrical property or a parameter related thereto of the effluent sample.

In one embodiment, the at least one pair of electrodes comprises a pair of electrodes that is configured to both create and electrical field in the effluent sample and sense the electrical property or a parameter related thereto of the effluent sample.

In one embodiment, the electrical system further comprises a third electrode and a fourth electrode.

In one embodiment, the at least one pair of electrodes comprises a pair of electrodes that is configured to induce electrical current into the effluent sample, and another pair of electrodes that is configured to sense the electrical property of the effluent sample.

In one embodiment, the processor is further configured to operate an algorithm configured to collectively process the optical signal and the property signal to determine an amount of a compound in the effluent sample.

In one embodiment, the compound is selected from a group consisting of leukocytes, blood cells, proteins, fat particles, triglycerides, chylomicrons, micelles, biological materials, and derivatives thereof.

In one embodiment, the system is integrated with an electromechanical cycler used for PD.

In one embodiment, the system is incorporated within the electromechanical cycler.

In one embodiment, the container is a portion of a first tube within the electromechanical cycler.

In one embodiment, the system is connected to a second tube connected to the patient and configured to drain effluent fluid from the peritoneal cavity of the patient and pass it through the container.

In one embodiment, the container is a portion of the second tube.

In one embodiment, the system communicates measured results to the electromechanical cycler.

A second aspect of the present disclosure is a system for characterizing an effluent sample from a patient undergoing peritoneal dialysis (PD). The system of the second aspect includes a container, an electrical system, an optical system, and a processor. The container encloses the effluent sample. The electrical system comprises a first pair of electrodes and a second pair of electrodes, with both the first pair of electrodes and the second pair of electrodes comprising a portion that is optically transparent, attached to the container, and configured to measure an electrical property of the effluent sample. The optical system comprises a light source and a photodetector. The light source is configured to emit a beam of radiation that passes through the first pair of electrodes and irradiates the effluent sample. The photodetector is configured to detect the radiation after it irradiates the effluent sample and passes through the second pair of electrodes to generate an optical property of the effluent sample. The processor operates an algorithm configured to collectively process the optical property and the electrical property to characterize the effluent sample.

A third aspect of the present disclosure is a system for measuring leukocytes from an effluent sample from a patient undergoing peritoneal dialysis (PD). The system of the third includes a container, an electrical system, an optical system, and a processor. The container encloses the effluent sample. The electrical system comprises a first pair of electrodes and a the second pair of electrodes, with both the first pair of electrodes and second pair of electrodes being optically transparent and attached to the container, with the first pair of electrodes configured to induce an electrical current into the effluent sample, and the second pair of electrodes configured to measure an electrical signal of the effluent sample that depends on the electrical current that is induced into the sample. The optical system comprises a light source and a photodetector. The light source is configured to emit a beam of radiation that passes through the one of the first pair of electrodes and second pair of electrodes and irradiates the effluent sample. The photodetector is configured to detect the radiation after it irradiates the effluent sample and passes through one of the first pair of electrodes and second pair of electrodes to generate an optical signal. The processor operates an algorithm configured to collectively process the optical signal and the electrical signal to characterize the effluent sample.

A fourth aspect of the present disclosure is a system for characterizing a liquid sample. The system of the fourth aspect includes a container, an optical system, an electrical system, and a processor. The container encloses the sample. The optical system comprises a light source and a photodetector. The light source is configured to emit a beam of radiation that passes through the container and irradiates the sample. The photodetector configured to detect the radiation after it irradiates the sample to generate an optical signal. The electrical system comprises a at least one pair of electrodes, wherein the at least one pair of electrodes is attached to the container and configured to measure an electrical property of the sample to generate a property signal. The processor operates an algorithm configured to collectively process the optical signal and the property signal to characterize the sample.

Additional features and advantages of the disclosed devices, systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been principally selected for readability and instructional purposes, and not to limit the scope of the subject matter.

### BRIEF DESCRIPTION OF THE DRAWINGS

Understanding that the figures depict only typical embodiments and are not to be considered to be limiting the scope of the present disclosure, the present disclosure is described and explained with additional specificity and detail through the use of the accompanying figures. The figures are listed below.

FIG. 1 is a schematic drawing showing a PDEAS according to an embodiment integrated with a PD machine that, in turn, connects to a patient;

FIG. 2 is a schematic drawing showing the PDEAS of FIG. 1;

FIG. 3A is a mechanical drawing showing a sample cell used to enclose PD effluent in the PDEAS of FIG. 2;

FIG. 3B is a schematic drawing showing pairs of sense and drive electrodes used for an impedance measurement in the sample cell of FIG. 3A;

FIGS. 3C-D are photographs of the sample cell of FIG. 3A, respectively, by itself and integrated with a circuit board controlling both impedance and optical measurements;

FIGS. 4A-C are mechanical drawings of an alternate sample cell according to an embodiment shown, respectively, from the front, bottom, and side of the cell having both drive and sense capabilities for a single pair of electrodes;

FIG. 5 is a photograph of a piece of tubing used to transport PD effluent from the PDEAS that is compressed and used during an optical measurement according to an embodiment;

FIGS. 6A to 6B are plots of optical transmission vs. the relative concentration of white blood cells, measured at λ=415 nm wavelength and λ=910 nm wavelength, respectively;

FIG. 7 illustrates measured absorption across different wavelengths with a CCD-based optical spectrometer and an AS7262 spectral sensor;

FIG. 8 is a plot of optical transmission vs. time as measured with an optical system used in the PDEAS from a sample of yeast cells dissolved in an aqueous solution;

FIG. 9A is a collection of plots of optical absorption vs. frequency as measured with an optical system used in the PDEAS from a sample of blood dissolved in an aqueous solution;

FIG. 9B is a plot of the magnitude of an absorption peak measured at λ=600 nm vs. the relative concentration of blood dilution wherein data for the plot were extracted from the graphs in FIG. 9A;

FIG. 10 is a plot of transmitted laser current and scattered light current vs. the percentage of milk diluted in an aqueous solution as measured with an optical system used in the PDEAS;

FIG. 11A is a collection of plots of optical absorption vs. wavelength as measured with an optical system used in the PDEAS from a sample of yeast dissolved in aqueous solution;

FIG. 11B is a plot of optical absorption vs. the relative concentration of yeast, measured at λ=688 nm wavelength;

FIG. 12 is a plot of series resistance vs. frequency as measured with an impedance system used in the PDEAS from a sample of yeast dissolved in an aqueous solution;

FIG. 13 is a plot of series capacitance vs. frequency as measured with an impedance system used in the PDEAS from a sample of yeast dissolved in an aqueous solution;

FIG. 14A is a plot of impedance vs. the relative concentration of yeast cells wherein data for the plot were extracted from graphs similar to FIG. 12; and

FIG. 14B is a plot of series capacitance vs. the relative concentration of yeast cells wherein data for the plot were extracted from graphs similar to FIG. 13.

### DETAILED DESCRIPTION

The detailed description is to be construed as exemplary only; it does not describe every possible embodiment, as this would be impractical, if not impossible. One ordinary skill in the art could implement numerous alternate embodiments, which would still fall within the scope of the claims.

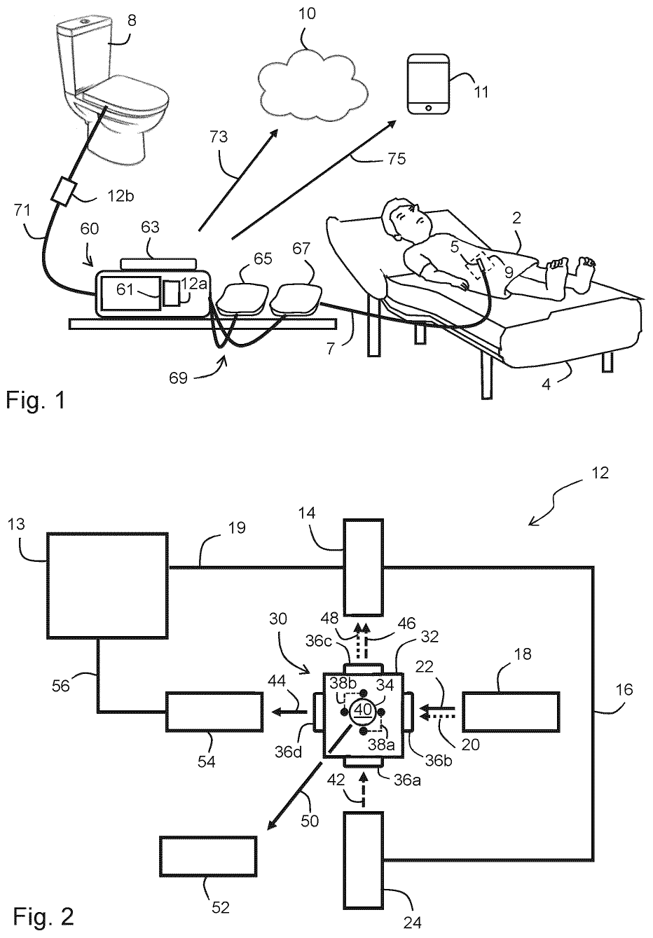

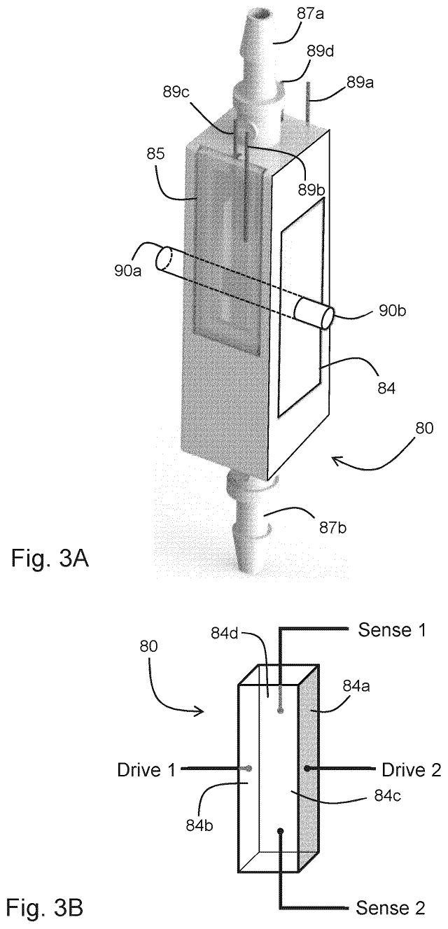

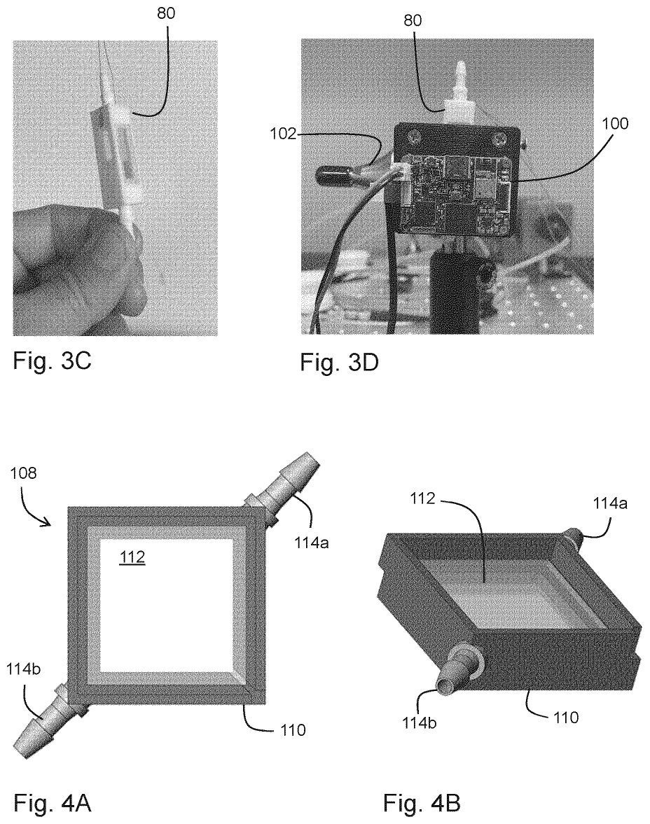

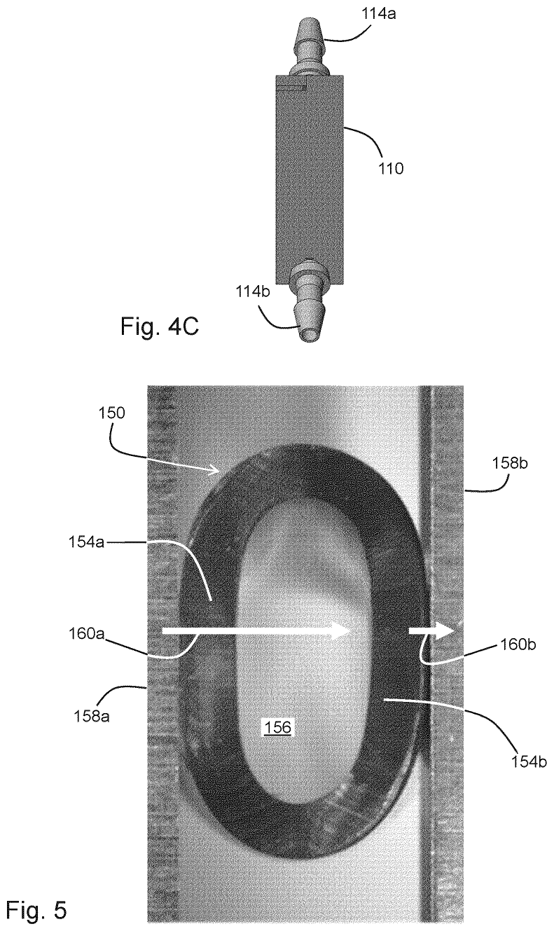

FIG. 1 illustrates a schematic drawing showing multiple PDEAS 12a, 12b integrated with a PD cycler 60 to measure PD effluent from a patient 2, with the specific goal of characterizing an amount of white blood cells in the effluent, which may indicate peritonitis. The PDEAS 12a. 12b may be incorporated within the PD cycler 60 (as depicted by PDEAS 12a), attached to tubing 71 that transports PD effluent to the patient's toilet 8 (as depicted by PDEAS 12b), or in any or portion of the PD cycler 60 that enclose PD effluent. While multiple PDEAS 12a, 12b are illustrated herein, it should be appreciated that only one PDEAS is necessary to measure PD effluent.

In each case, the PDEAS 12a, 12b is a compact measurement system that performs one or more of the following measurements from the PD effluent: i) optical spectroscopy; ii) optical scattering, and more typically laser scattering; iii) single or multi-frequency bio-impedance, and iv) optical transmission.

Optical measurement can include, for example, focused transmitted light for particle concentration and/or turbidity estimation, a ratio comparison of transmitted light, small angle scattered light, and large angle scattered light at a single wavelength, or a ratio comparison of any combination of transmitted and/or scattered light with different wavelengths of light, for the assessment of particle size, spectroscopy for determining type of particles by color (e.g. red/white blood cells, proteins, fat, and the like), and other similar optical measurements.

By combining several optical sensors, different optical properties can be distinguished from one another. For example, estimation of particle concentration (e.g. white blood cell) can be achieved by use of focused transmitted light. Cells will pass through the beam and interact with this light (e.g., absorbing, lensing, etc.), yielding changes in the measured transmitted intensity. Estimation of particle size can be achieved by measuring transmitted light and/or scattered light at several different angles. Particles at the size of cells scatter mostly at small angles (e.g., 0-10 degrees), whereas small particles, with diameter less than the wavelength of light, scatter at larger angles. White blood cells and red blood cells are typically larger than the wavelength of visible light and can be assumed to scatter light according to Mie scattering, whereas chylomicrons and cholesterol (e.g., VLDL. LDL, and HDL) are smaller than the wavelength of light, and scatter according to Rayleigh. A similar estimation of particle size can be established by using multiple wavelengths of light across multiple sensors; for example, a cell-sized particle is more likely to scatter light at a larger angle using a longer wavelength (e.g., infrared) than a shorter wavelength (e.g., ultraviolet).

By measuring light at, for example, 0, 5 and 20 degrees, when compared to the incident focused laser light beam, the 0 degree (i.e., transmitted light) can be used to estimate the number of blood cells and the ratios between 0, 5, and 20 degrees can be used to estimate the size of the particles, and, thus, if the cloudiness of effluent is mainly caused by blood cells or by lipoproteins. In an embodiment, detection angles of greater size, such as 45 or 90 degrees, can be used to estimate the presence of smaller and smaller particles. By determining particle size within the effluent, the effluent can be characterized (as detailed below in Table 1):

TABLE 1
Sizes of particles available in human plasma and
peritoneal fluid. As comparison the wavelength
of visible light is between 0.5 and 0.7 μm.

| Particle | Diameter (μm) |
|---|---|
| Erythrocytes (red blood cell) | 6-8 |
| White blood cells: | |
| Neutrophils | 10-12 |
| Eusinophils | 10-12 |
| Basophil | 12-15 |
| Small lymphocytes | 7-8 |
| Large lymphocytes | 12-15 |
| Monocytes | 15-30 |
| Lipoproteins: | |
| Chylomicrons | 0.1-1 |
| VLDL | 0.03-0.09 |
| LDL | 0.02-0.025 |
| HDL | 0.01-0.02 |

Continuing on, a computational system, located either within the PDEAS 12a. 12b, in a gateway 11 (e.g. a mobile phone), or in the cloud 10, analyzes information from these measurements to estimate both a concentration of leukocytes (e.g., white blood cells) within the PD effluent and the actual color of the PD effluent. The computational system further analyzes this information, in turn, to determine an early onset of an infection, e.g. peritonitis. Once this condition is detected, a clinician, for example, may provide the patient antibiotics or other medication to address this condition.

For example, by incorporating both a white light source and a photo spectrometer, the color of the absorbed light can be measured. This can be used to determine the color of the effluent and thus determine if the estimated number of cells counted by the focused transmitted light are caused by red or white blood cells. The color of the effluent can additionally be used to detect other complications.

In order for the PDEAS 12a, 12b to receive PD effluent, the PD cycler 60 attaches to the patient 2 through a first tube 7. The tube 7 connects to a catheter 5 that is sutured into the patient's peritoneum using a simple surgical procedure. The catheter 5, for example, may protrude out from an opening 9 in a garment worn by the patient 2 lying on bed 4. Attached to the PD cycler 60 through a collection of tubes 69 are bags 65, 67 containing different mixtures of PD dialysate.

During therapy, typically 2-3 liters of dialysate flows through the collection of tubes 69 and catheter 5, and finally into the patient's peritoneum to form the fill volume. The fill volume remains in the abdomen and waste products diffuse across the peritoneum from the underlying blood vessels.

After a variable period of time (usually 2-6 hours depending on the treatment), PD effluent flows out through the catheter, through the collection of tubes, and into the PD cycler, where it is measured with the PDEAS 12a. 12b as described above to detect a concentration of leukocytes or other fluid conditions. The PD cycler 60 may additionally include standard components such as a heating system 63 to warm the bags 65, 67 to the patient's body temperature, a display 61 allowing the patient 2 or a user associated with the patient to control the PD cycler 60, and an internal wireless module configured to transmit information from the PDEAS 12a. 12b to either the gateway 11 (as indicated by arrow 75) or cloud (as indicated by arrow 73).

FIG. 2 illustrates a schematic drawing showing the PDEAS 12 in greater detail. Generally, it should be appreciated that this system, as described in greater detail below, includes optical and electrical sensors to measure the PD effluent as it passes through a cuvette 34 enclosed by an opening 40 in sample holder 30 (also referred to herein as measurement cell 30). Additionally, while FIG. 2 illustrates multiple optical and electrical sensors within the PDEAS 12, it is understood that one or all of these may be used to measure the PD effluent to determine its properties, e.g. its color and concentration of white blood cells, or electrical characteristics.

The PDEAS 12 includes a computer processing unit 13, along with a customized circuit board. The circuit board may advantageously include an optical detector 14. The optical detector 14 is electrically coupled to a laser light source 24 via electrical connection 16 and is electrically coupled to computer processing unit 13 via electrical connection 19. For example, laser light source 24 is a narrow band laser light source that outputs laser radiation 42 at a given wavelength.

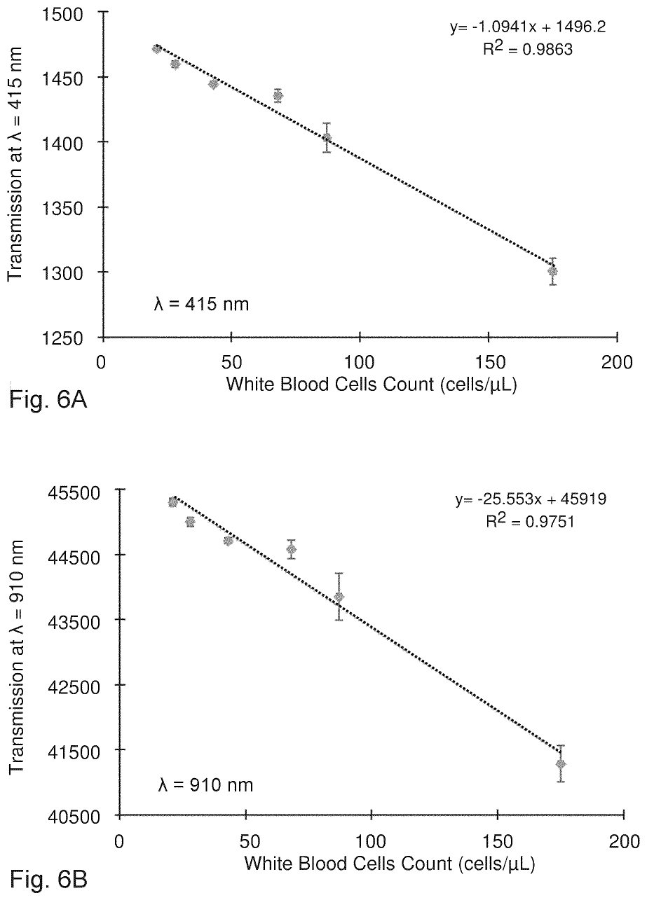

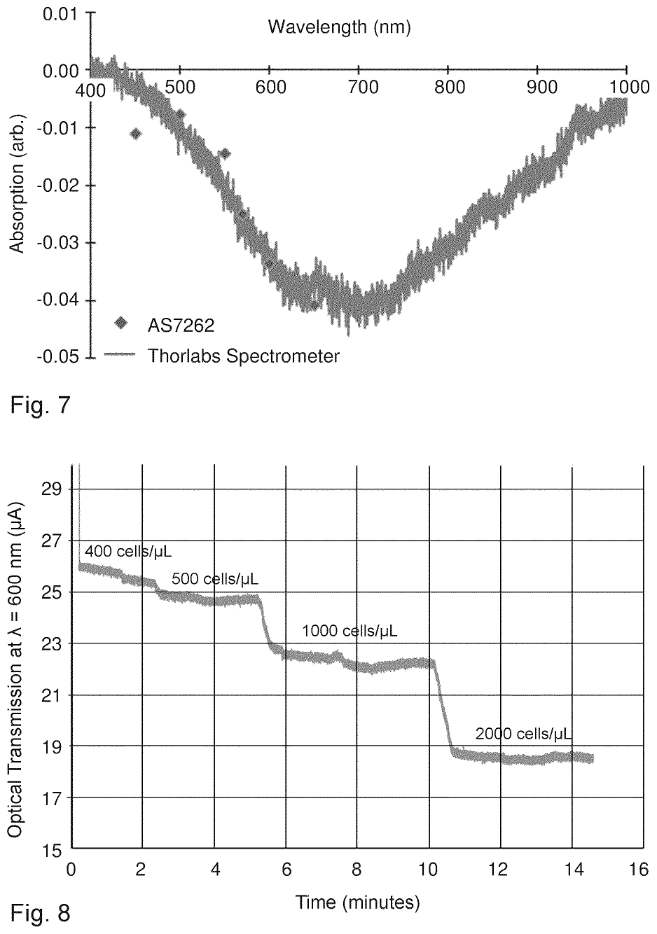

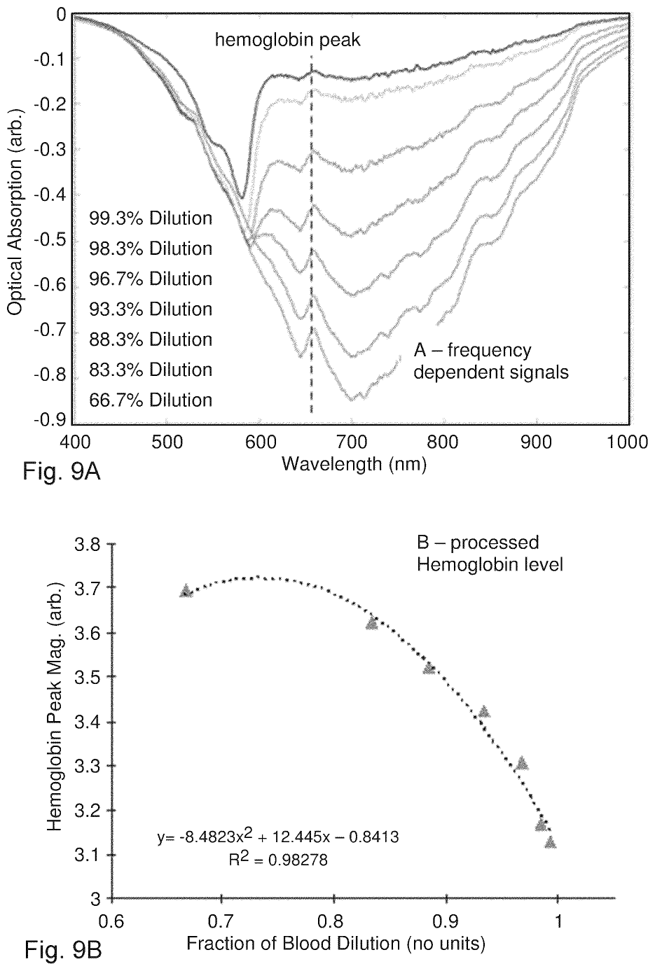

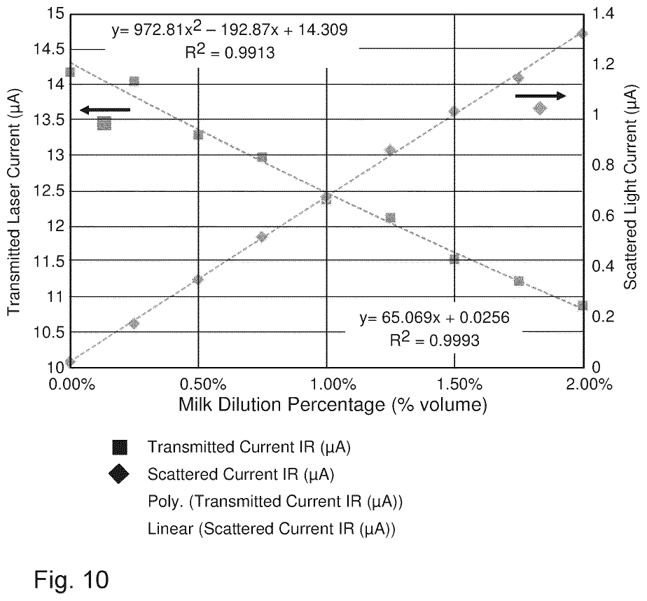

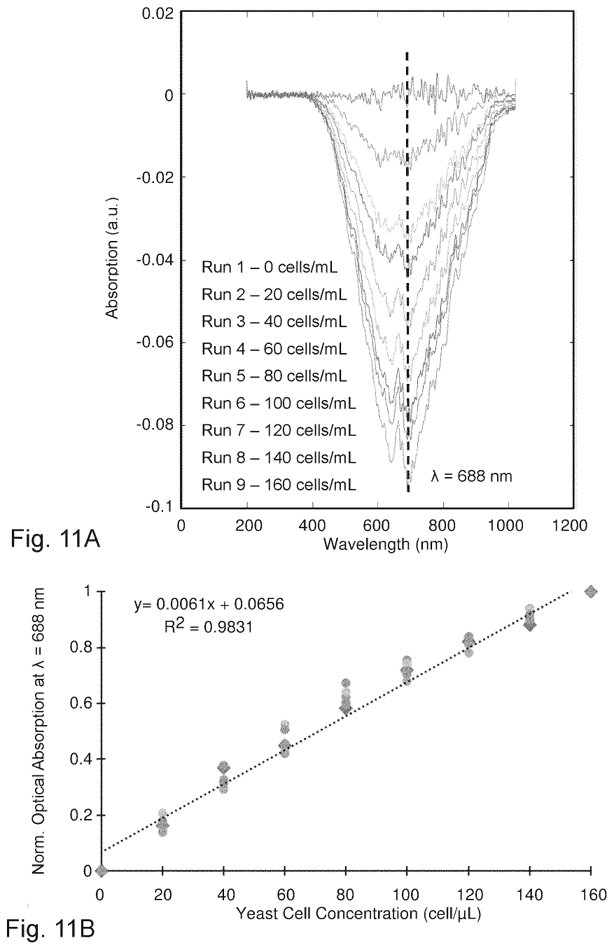

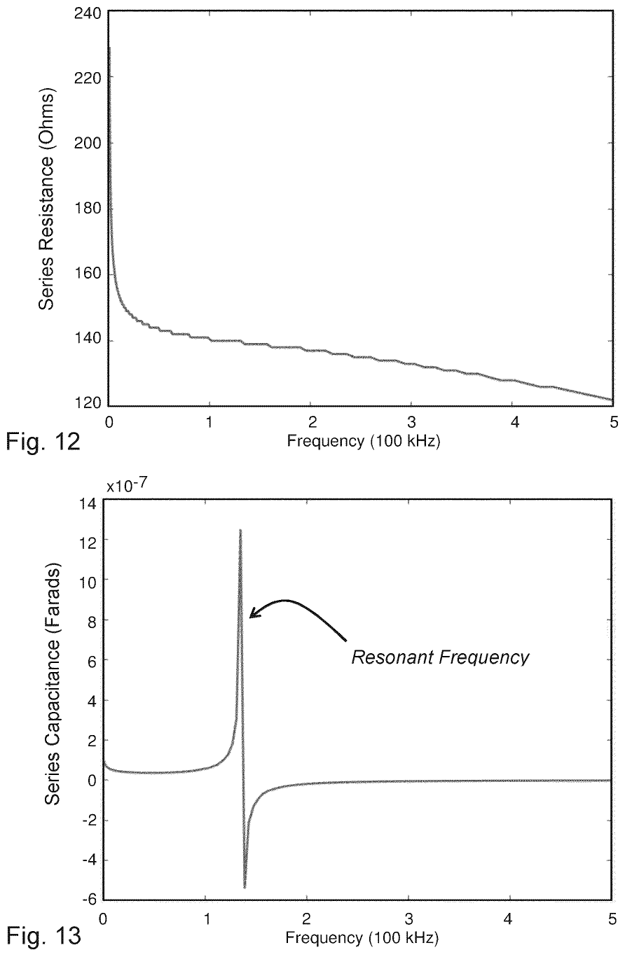

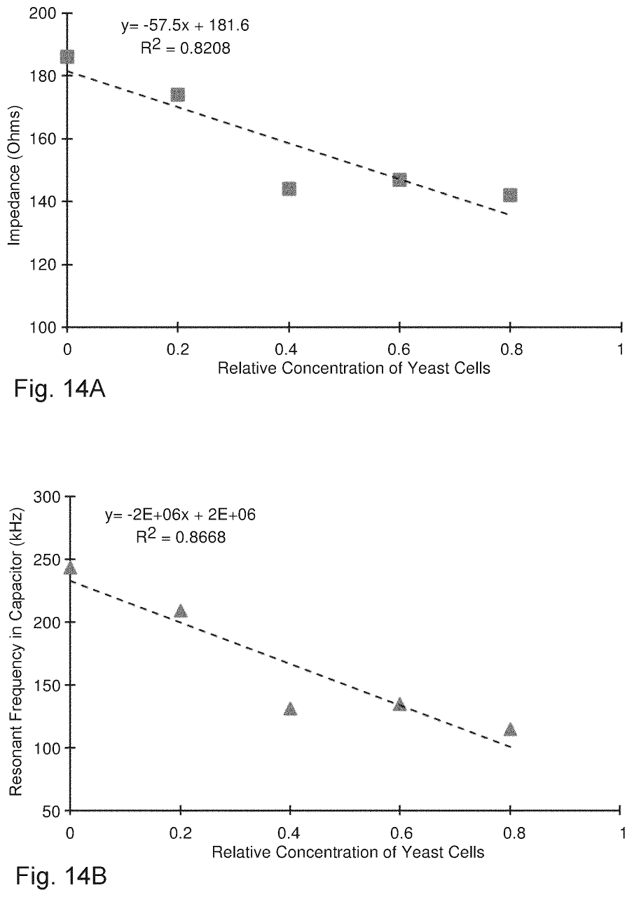

PDEAS 12 may further include a light source 18 with optical detector 54, such as a broadband optical detector and/or segmented/multiple narrow band detectors, configured to measure an optical spectrum (similar to that shown in FIGS. 6A to 6B) from the PD effluent. For example, light source 18 is a white LED. In the illustrated example, the PDEAS 12 includes two separate light sources, laser light source 24 and light source 18. These two separate light sources are each configured to pass light (visible or otherwise) through PD effluent as described in greater detail herein. These two separate light sources may each be configured to be detected independently, such that two separate light source readings may be readily determined. Lastly, these two separate light sources may be configured to output a single coherent wavelength or, alternatively, be configured to output different light sources (e.g., different wavelengths, such as red, green, or infrared, different intensities, and the like) to provide a multitude of light source readings across the spectrum.

As illustrated in FIG. 2, laser light source 24 and optical detector 14 are oriented orthogonal to light source 18 and detector 54. It should be appreciated that other orientations are, likewise, contemplated herein.

Light source 18 may be configured to output multiple different iterations of light (e.g., different wavelengths, different intensities, and the like). For example, light source 18 outputs a first set of radiation 20 (at a first wavelength) and also outputs a second set of radiation 22 (at a second wavelength).

Thus, as illustrated by FIG. 2, a total of three different radiations are output toward a measurement cell 30: laser radiation 42, first set of radiation 20, and second set of radiation 22. Each of these three radiations are configured to pass through measurement cell 30.

Namely, measurement cell 30 includes a metal holder 32 and an optically transparent cuvette 34 (e.g., a glass sample cell), which surrounds PD effluent flowing through opening 40. Thus, laser radiation 42, first set of radiation 20, and second set of radiation 22 are configured to pass through transparent cuvette 34 and PD effluent flowing through opening 40. PDEAS 12 further includes a lensing system 36a-d. For example, PDEAS 12 includes a first set of lenses 36a-b, which are configured to receive one or more of laser radiation 42, first set of radiation 20, and second set of radiation 22. Similarly, PDEAS 12 includes a second set of lenses 36c-d, which are configured to receive one or more of laser radiation 42, first set of radiation 20, and second set of radiation 22 after the radiation passes through optically transparent cuvette 34 and PD effluent flowing through opening 40.

PDEAS 12 further includes a first set of sense/drive electrodes 38a and a second set of sense/drive electrodes 38b. For example, the first set of sense/drive electrodes 38a are configured to induce current into PD effluent flowing through opening 40; similarly, for example, the second set of sense/drive electrodes 38b are configured to detect current (induced from first set 38a) from PD effluent flowing through opening 40. Generally, via first set of sense/drive electrodes 38a and second set of sense/drive electrodes 38b. PDEAS 12 is capable of taking impedance measurements of PD effluent flowing through opening 40.

Each of first set of sense/drive electrodes 38a and second set of sense/drive electrodes 38b may be electrically coupled to an impedance circuit 52 (e.g., via electrical connection 50).

1-42. (canceled)

43. A system for characterizing an effluent sample from a patient undergoing peritoneal dialysis, PD, comprising:
a container for enclosing the effluent sample;
an optical system comprising a light source and a photodetector, the light source configured to emit a beam of radiation that passes through the container and irradiates the effluent sample, and the photodetector configured to detect the radiation after it irradiates the effluent sample to generate an optical signal; and
a processor operating an algorithm configured to process the optical signal to characterize the effluent sample.

44. The system of claim 43, further comprising an electrical system comprising at least one pair of electrodes, wherein the at least one pair of electrodes is attached to the container and configured to measure an electrical property of the effluent sample to generate a property signal, and wherein the algorithm is configured to collectively process the optical signal and the property signal to characterize the effluent sample.

45. The system of claim 44, wherein the processor is further configured to operate the algorithm configured to collectively process the optical signal and the property signal to determine an amount of a compound in the effluent sample.

46. The system of claim 45, wherein the compound is selected from a group consisting of leukocytes, blood cells, proteins, fat particles, triglycerides, chylomicrons, micelles, biological materials, and derivatives thereof.

47. The system of claim 43, which is integrated with an electromechanical cycler used for PD.

48. The system of claim 47, which is incorporated within the electromechanical cycler.

49. The system of claim 47, wherein the container is a portion of a first tube within the electromechanical cycler.

50. The system of claim 47, which is connected to a second tube connected to the patient and configured to drain effluent fluid from the peritoneal cavity of the patient and pass it through the container.

51. The system of claim 50, wherein the container is a portion of the second tube.

52. The system of claim 43, which is configured to communicate measured results to the electromechanical cycler.

53. The system of claim 43, wherein the processor is configured to process the optical signal for determination of at least one of particle concentration, particle size, particle type, or color of the effluent sample.

54. The system of claim 44, wherein the processor is configured to process the property signal for determination of at least one of particle concentration or particle type.

55. The system of claim 53, wherein the optical system is configured, by the light source, to emit broadband radiation and, by the photodetector, to detect the broadband radiation after it has passed through the effluent sample, for determination of an optical spectrum representing the effluent sample.

56. The system of claim 55, wherein the processor is configured to process the optical signal, representing the optical spectrum, for determination of at least one of color, particle type, or particle concentration of the effluent sample.

57. The system of claim 55, wherein the optical system is configured, by the light source, to emit the broadband radiation and, by the photodetector, to detect the broadband radiation after it has passed through a reference sample of a fluid other than the effluent, for determination of a reference spectrum representing the reference sample, and wherein the processor is configured to determine said at least one of color, particle type, or particle concentration of the effluent sample based on a difference between the optical spectrum and the reference spectrum.

58. The system of claim 53, wherein the optical system is configured, by the light source, to emit narrowband radiation and, by the photodetector, to detect the narrowband radiation after it has passed through the effluent sample, for determination of an optical transmission value for the effluent sample, and/or to detect the narrowband radiation at one or more transverse angles to the beam of radiation after it has been scattered by the effluent sample, for determination of a scattering magnitude at the one or more transverse angles.

59. The system of claim 58, wherein the light source is configured to emit, in sequence, the narrowband radiation at two or more wavelengths for determination of at least one of the optical transmission value or the scattering magnitude, at each of the two or more wavelengths.

60. The system of claim 58, wherein the processor is configured to process the optical transmission value and/or the scattering magnitude for determination of at least one particle size in the effluent sample.

61. The system of claim 60, wherein the processor is configured to determine at least one particle type in the effluent sample based on the at least one particle size.

62. The system of claim 61, wherein the processor is further configured to determine a particle concentration of the at least one particle type in the effluent sample based on the optical transmission value and/or the scattering magnitude.

63. The system of claim 53, wherein the particle type is selected from a group consisting of leukocytes, blood cells, proteins, fat particles, triglycerides, chylomicrons, micelles, biological materials, and derivatives thereof.
